(19) United States
(12) Patent Application Publication
(10) Pub. No.: US 2004/0028635 A1
(43) Pub. Date: Feb. 12, 2004

(54) Block-structure copolymer consisting of a saccharide segment bound to at least a biodegradable hydrophobic segment, and corresponding particles

(76) Inventors: Cedric Chauvierre, Chatenay-Malabry (FR); Patrick Couvreur, Sur-Yvette (FR); Denis Labarre, Villebon-Sur-Yvette (FR); Christine Vauthier, Sur-Yvette (FR)

Correspondence Address: YOUNG & THOMPSON, 745 SOUTH 23RD STREET 2ND FLOOR, ARLINGTON, VA, 22202

(21) Appl. No.: 10/416,840

(22) PCT Filed: Nov. 16, 2001
(86) PCT No.: PCT/FR01/03619

(30) Foreign Application Priority Data
Nov. 17, 2000 (FR) 00/14900

(51) Int. Cl. A61K 7/06; A61K 7/11; C08G 63/48; C08G 63/91

(52) U.S. Cl. 424/70.13; 525/54.2

(57) ABSTRACT

A block-structure copolymer consisting of a hydrophilic saccharide segment and at least a biodegradable hydrophobic segment of general formula (I),

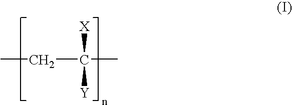

wherein: X represents a CN or CONHR radical; Y represents a COOR′, CONHR″ radical with R, R′ and R″ representing, independently of each other, a hydrogen atom, a linear or branched C₁-C₂₀ alkyl group, a linear or branched C₁-C₂₀ alkoxy group, an amino acid radical, a mono-hydroxylated or polyhydroxylated radical or a C₅-C₁₂ aryl or heteroaryl radical, the saccharide segment being bound either by one of its ends to a single segment of general formula (I), or by each of its two ends, to a segment of general formula (I), the two hydrophobic segments being identical or different. The invention also concerns particles based on the copolymer and a corresponding preparation method.

The invention relates to a novel family of biodegradable copolymers based on a polymer of alkyl cyanoacrylate or related type and on poly- or oligosaccharides, which are particularly useful in the pharmaceutical, veterinary, food-processing and cosmetic fields, in particular as vehicles and/or excipients. It also provides a process for preparing these copolymers.

The biodegradable polymers generally formulated under the appearance of liposomes, microemulsions, nanospheres, nanocapsules, microspheres, microcapsules, microparticles and nanoparticles constitute efficient delivery systems for active principles. Among these systems, micro- and nanoparticles based on poly(alkyl cyanoacrylate) have proved to be very particularly advantageous due to their rapid bioerosion in comparison with other biodegradable polymers, such as poly(lactic acid)/poly(ε-caprolactone), for example.

However, these particles based on poly(alkyl cyanoacrylate) are not entirely satisfactory. On the one hand, there exists today no efficient preparation method for obtaining these systems with a controlled structure and/or composition of the polymers participating in their formation. Furthermore, these particles have the disadvantage of being rapidly captured by the macrophages of the Mononuclear Phagocyte System (MPS). Their lifetime in vivo is therefore very short.

The present invention is targeted specifically at compensating for the abovementioned disadvantages and at providing a novel material for particles, the polymer structure of which derives from the combination with a polymer related to poly(alkyl cyanoacrylate) of a segment of poly- or oligosaccharide nature, such as dextran, for example.

Nanoparticles based on amphiphilic block copolymers comprising dextran and poly(alkyl cyanoacrylate) segments have already been described (S. J. Douglas et al.; Journal of Controlled Release (1986), 15-23). However, these copolymers, which derive from the anionic polymerization of cyanoacrylate monomers in the presence of dextran, have grafted structures. This is because the polymerization by the anionic route of alkyl cyanoacrylates in the presence of dextran results in the grafting of several poly(cyanoacrylate) chains to the dextran polymer chain, without, furthermore, it being possible to control the number, the size and the location of these poly(cyanoacrylate) chains. Finally, the particles thus formed have, at the surface, an interfacial layer of dextran, the overall structure of which results in recognition by the complement system and by the macrophages of the MPS.

The present invention is targeted, for its part, at providing copolymers deriving from cyanoacrylate or equivalent monomers and from oligo- or polysaccharides but having a completely different structure. Thus it is that, in the context of the present invention, the copolymers are provided in a block form, in contrast to the grafted forms described above. This block form is in fact inaccessible by the anionic polymerization route discussed above.

The present invention is also targeted at providing a synthetic route for preparing block copolymers of this type.

The inventors have thus demonstrated that it is possible to efficiently polymerize by the radical route molecules with a high charge density, of cyanoacrylate type, in the presence of poly- or oligosaccharides, this being despite the fact that the activation energy necessary for this radical polymerization is much greater than that necessary by anionic polymerization. This is because, because of their high charge density, monomers of cyanoacrylate or equivalent type are naturally inclined to generate their anionic form when they are brought into the presence of a nucleophilic agent, such as, for example, OH⁻ anions, and thus to polymerize by the anionic route.

In point of fact, the inventors have demonstrated that it is possible, first, to effectively slow down the appearance of this polymerization, in particular through the control of the pH of the polymerization medium, and, secondly, to favor the polymerization by the radical route. Finally, against every expectation, the radical polymerization route provided by the inventors proves to be faster than the anionic polymerization.

Consequently, a first subject matter of the present invention is a copolymer comprising a block structure composed of a hydrophilic segment of saccharide nature, at least one of the ends of which is bonded to a biodegradable hydrophobic segment of general formula (I):

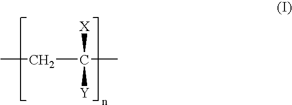

in which:

X represents a CN or CONHR radical,

Y represents a COOR′ or COHNR″ radical,

with R, R′ and R″ representing, independently of one another, a hydrogen atom, a linear or branched C₁ to C₂₀ alkyl group, a linear or branched C₁ to C₂₀ alkoxy group, an amino acid radical, a mono- or polyhydroxylated acid radical or a C₅ to C₁₂ aryl or heteroaryl radical,

with said segment of saccharide nature being bonded either by one of its ends to a single segment of general formula (I) or by each of its two ends to a segment of general formula (I), the two hydrophobic segments being identical or different.

In the segment of general formula (I), X preferably represents a CN radical. More preferably, Y represents a COOR′ radical with R′ as defined above.

The repeat unit of isobutyl cyanoacrylate can be more particularly mentioned by way of illustration of a unit capable of composing a segment of general formula (I).

The term “block structure” is intended to denote, according to the invention, a structure which derives from the establishment of a covalent bond between at least one of the ends of the segment of saccharide nature and one of the ends of the polymer chain of general formula (I). The field of the invention thus encompasses copolymer structures comprising either a single segment of general formula (I) bonded to one end of the segment of saccharide nature or two identical or different segments of general formula (I) bonded respectively on either side of the segment of saccharide nature. In contrast to the grafted structures mentioned above, the claimed copolymers do not have side branch(es) of saccharide nature on the hydrophobic segment or side branch(es) of hydrophobic nature on the segment of saccharide nature.

The covalent bond established between the two types of segment is generally of C—C or C—O—C nature. It preferably derives from the radical polymerization of at least one molecule of a compound of formula (II):

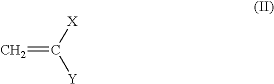

in which X and Y are as defined above, in the presence of a poly- or oligosaccharide.

This radical polymerization is preferably carried out under the conditions set out below for the claimed process.

In particular, it is carried out at pH and atmosphere conditions unfavorable to the presence and/or to the generation of anions in the reaction medium and in the presence of a sufficient amount of a suitable redox radical initiator.

The segment of saccharide nature derives from an oligo- or polysaccharide of natural or synthetic origin which may or may not have been modified.

The term “modified polysaccharide” is understood to mean any polysaccharide which has undergone a change on its backbone, such as, for example, the introduction of reactive functional groups or the grafting of chemical entities (molecules, aliphatic links, PEG chains, and the like). Polysaccharides modifed by grafting biotin, fluorescent compounds, and the like, are available commercially. Other polysaccharides grafted with hydrophilic chains (for example, PEG) have been described in the literature. It is also possible to envisage using, in the context of the present invention, polysaccharides modified like those described in the reference Jozefowicz and Jozefonvicz, Biomaterials, 18, 1633-1644 (1997). Of course, this modification must not affect the polymerization of the monomer of general formula (II) in the presence of the modified oligo- or polysaccharide.

According to a preferred alternative form of the invention, the oligo- or polysaccharide employed according to the invention may already per se possess biological properties and/or activities. For example, it may confer anticoagulant, vaccinating or targeting properties or even masking properties, to prevent capture by the macrophages of the MPS.

Thus it is that it can be:

oligo- or polysaccharides exhibiting antigenic properties, such as, for example, those of bacterial or viral origin,

oligo- or polysaccharides possessing biological activity, such as, for example, heparin, heparan sulfate, dermatan sulfate, dextran sulfate and pentosan sulfate, dextran substituted by carboxyl and sulfate or sulfonate groups, sulfated polysaccharides extracted from algae (fucans and fucoidans), poly(sialic acid)s or sulfated hyaluronic acid, which possess anticoagulant activities or antiinflammatory activities, to variable extents, and/or

oligo- or polysaccharides which are involved in cell recognition and cell signaling processes, such as, for example, poly(sialic acid)s, heparin sulfate, blood group antigens, polysaccharides and lipopolysaccharides of various bacterial strains, oligosaccharide chains of membrane and/or circulating glycoproteins, and oligosaccharide chains of glycolipids.

The copolymers deriving from polysaccharides of this type prove, of course, to be particularly advantageous in terms of therapeutic use insofar as they naturally possess an intrinsic biological activity and thus can be used as such on this account.

The polysaccharides which are very particularly suitable in the invention are or derive from D-glucose (cellulose, starch, dextran, cyclodextrin), D-galactose, D-mannose, D-fructose (galactosan, mannan, fructosan) or fucose (fucan). The majority of these polysaccharides comprise the elements carbon, oxygen and hydrogen. The polysaccharides in accordance with the invention can also comprise sulfur and/or nitrogen. They can thus derive from glycoprotein or from glycolipid. Likewise, hyaluronic acid (composed of N-acetylglucosamine and glucuronic acid units), poly(sialic acid), also known as colominic acid or poly(N-acetylneuraminic acid), chitosan, chitin, heparin or orosomucoid comprise nitrogen, while agar, a polysaccharide extracted from marine algae, comprises sulfur in the form of hydrogen sulfate (>CH—O—SO₃H). Chondroitin sulfuric acid and heparin comprise both sulfur and nitrogen.

According to a preferred alternative form of the invention, the polysaccharide has a molecular weight of greater than or equal to 6000 g/mol.

In the specific case of dextran and of amylose (C₆H₁₀O₅)ₙ, n varies between 10 and 620 and preferably between 33 and 220. In the case of hyaluronic acid, the molar mass varies between 5×10³ and 5×106 g/mol, preferably between 5×1 and 2×10⁶ g/mol. In the case of chitosan, the molar mass varies between 6×10³ and 6×10⁵ g/mol, preferably between 6×10³ and 15×10⁴ g/mol.

Mention may be made, as illustration of the polysaccharides which are more particularly suitable in the invention, of polydextroses, such as dextran, chitosan, pullulan, starch, amylose, cyclodextrins, hyaluronic acid, heparin, amylopectin, cellulose, pectin, alginate, curdlan, fucan, succinoglycan, chitin, xylan, xanthan, arabinan, carrageenan, poly(glucuronic acid), poly(N-acetylneuraminic acid), poly(mannuronic acid) and their derivatives (such as, for example, dextran sulfate, amylose esters, cellulose acetate, pentosan sulfate, and the like).

In the specific case of a cyclic polysaccharide like cyclodextrins, its covalent coupling with a compound of general formula (II), during the radical polymerization, has the effect of bringing about opening of the ring and thus of leading the polysaccharide to adopt a linear structure in accordance with the invention.

Dextran, heparin, poly(N-acetylneuraminic acid), amylose, chitosan, pectin and hyaluronic acid, and their derivatives, are more particularly preferred.

The copolymers advantageously have a controlled content of oligo- or polysaccharide.

The claimed copolymer can be provided in a soluble form or under the appearance of a precipitate, of micelles or of particles. According to an advantageous aspect of the invention, it is provided under the appearance of particles. They can be micro- or nanoparticles.

In the specific case of particles and micelles, it is probable that the copolymer has a structure arranged as follows: the chains of the same nature, that is to say saccharide or hydrophobic chains, group together, either to form the core structure of the micelle or particle or the brush-like ring around this core structure. Their distribution between the core structure and the ring will, of course, depend on the nature, aqueous or organic, of the solvent in which the particles or micelles are dispersed. The term “brush-like ring” is intended to denote a structure in which the segments constituting the ring are bonded via one of their ends to the segments constituting the core. Their free ends constitute the periphery of the ring. Thus, in aqueous medium, the hydrophobic segments are grouped together so as to form the core and the segments of saccharide nature are positioned in a brush-like ring all around this core. In a solvent or organic type, this arrangement between the two types of segment is reversed: the core is of hydrophilic nature and is thus formed of the segments of saccharide nature and the brush-like ring is of hydrophobic nature and is thus formed of the segments of general formula (I).

In the case of the copolymers with a grafted structure described above, this brush-like ring structure cannot exist in an aqueous medium, insofar as several hydrophobic segments are covalently bonded to a single chain of saccharide nature.

A second aspect of the invention relates to particles composed of a copolymer in accordance with the invention.

Mention may more particularly be made, by way of representation of the claimed particles, of those composed of a copolymer deriving from the polymerization of:

isohexyl cyanoacrylate, isobutyl cyanoacrylate, n-butyl cyanoacrylate, n-propyl cyanoacrylate, ethyl cyanoacrylate or 2-methoxyethyl cyanoacrylate in the presence of dextran,

isobutyl cyanoacrylate in the presence of heparin, chitosan, pectin, hyaluronic acid, dextran sulfate or y-cyclodextrin.

The claimed particles can have a size of between 1 nm and 1 mm and preferably between 60 nm and 100 μm.

Generally, the particles having a size of between 1 and 1000 nm are then known as nanoparticles. Microparticles refer to particles with a size varying from 1 to several thousand microns.

These particles can, in some cases, be provided in an aggregated or micellar form. Thus it is that the particles resulting from the polymerization of isobutyl cyanoacrylate in the presence of γ-cyclodextrin have an aggregated appearance.

These particles can possess a biological activity, either because of the nature of the polysaccharide from which they are formed or because they additionally incorporate a biological or pharmaceutical active material.

Mention may more particularly be made, as biological active materials, of peptides, proteins, carbohydrates, nucleic acids, lipids, polysaccharides or their mixtures. They can also be synthetic organic or inorganic molecules which, administered in vivo to an animal or to a patient, are capable of inducing a biological effect and/or of manifesting a therapeutic activity. They can thus be antigens, enzymes, hormones, receptors, peptides, vitamins, minerals and/or steroids.

Mention may be made, by way of representation of the medicaments capable of being incorporated in these particles, of antiinflammatory compounds, anesthetics, chemotherapeutic agents, immunotoxins, immunosuppresants, steroids, antibiotics, antivirals, antifungals, antiparasitics, vaccinating substances, immunomodulators and analgesics.

Likewise, it is possible to envisage combining, with these active materials, compounds intended to participate with regard to their release profile. For example, PEG chains or polyester chains (which may or may not be modified) can be added to the composition of the particles and “composite” particles can thus be obtained.

It is also possible to incorporate, in the particles, compounds with a diagnostic purpose. They can thus be substances detectable by X-rays, fluorescence, ultrasound, nuclear magnetic resonance or radioactivity. The particles can thus include magnetic particles, radio-opaque materials (such as, for example, air or barium) or fluorescent compounds. For example, fluorescent compounds, such as rhodamine or nile red, can be included in particles with a hydrophobic core. Alternatively, gamma emitters (for example, indium or technetium) can be incorporated therein. Hydrophilic fluorescent compounds can also be charged to the particles but with a reduced efficiency in comparison with the hydrophobic compounds, because of their low affinity with the matrix.

Finally, these particles can be combined with peptides/proteins capable of helping them diffuse through biological membranes, such as the TAT peptide, or compounds such as the ZOT (Zonula occludens Toxin) protein and zonulin or equivalents, or any other absorption promoter.

In this case, this type of combination can be prepared by chemical functionalization of the polysaccharide surface of the particles. It is thus possible to envisage covalently attaching, at functional groups present on the backbone of saccharide nature, specific ligands, such as targeting agents, labels or, more generally, any compound capable of conferring on said particles a capability of reacting with an external species, such as, for example, a functional group on a support or a biological entity present in a medium under consideration.

Commercial magnetic particles having controlled surface properties can also be incorporated in the matrix of the particles or can be covalently attached to one of their constituents.

The active material can be incorporated in these particles during their process of formation or, in contrast, can be charged to the particles once the latter are obtained. It is thus possible to charge them by adsorption or by covalent grafting.

The particles according to the invention can be administered in different ways, for example, by the oral, parenteral, ocular, pulmonary, nasal, vaginal, cutaneous or buccal routes, and the like. The noninvasive oral route is a route of choice.

Another subject matter of the present invention is the use of the particles as vehicle for pharmaceutical, cosmetic, food-processing or veterinary active principles.

A third aspect of the present invention relates to a process for the preparation of the claimed copolymer.

More specifically, the present invention is targeted at a process of use in the preparation of block copolymers composed of a hydrophilic segment of saccharide nature, at least one of the ends of which is bonded to a hydrophobic segment, characterized in that it comprises the polymerization by the radical route of at least one molecule of a compound of general formula (II):

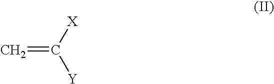

in which:

X represents a CN or CONHR radical,

Y represents a COOR′ or COHNR″ radical,

with R, R′ and R″ representing, independently of one another, a hydrogen atom, a linear or branched C₁ to C₂₀ alkyl group, a linear or branched C₁ to C₂₀ alkoxy group, an amino acid radical, a mono- or polyhydroxylated acid radical or a C₅ to C₁₂ aryl or heteroaryl radical,

said radical polymerization being carried out in the presence of at least one molecule of a poly- or oligosaccharide under pH and atmosphere conditions unfavorable to the presence and/or to the generation of anions in the reaction medium and in the presence of a sufficient amount of a suitable radical redox initiator.

Preferably, X represents a CN radical and/or, more preferably, Y represents a COOR radical.

As stated above, it proves to be possible to favor the radical polymerization, at the expense of the naturally predominant anionic polymerization, in particular by carrying out the polymerization reaction under pH conditions unfavorable to the generation of free anions, such as, for example, OH⁻ ions.

To do this, the pH of the reaction medium is preferably adjusted to a value of less than 2 and more preferably of less than 1.5.

Surprisingly, it appears that the adjustment of the pH to such a value was not harmful to the rate of polymerization. Against every expectation and as emerges from the examples presented below, the radical polymerization initiated under these pH conditions on the contrary takes place at a rate greater than that of an anionic polymerization. This is illustrated in particular by FIGS. 1 and 2.

According to a preferred alternative form of the invention, the reaction is also carried out under inert atmosphere conditions. The solvent is advantageously chosen so that, while maintaining conditions favorable to the radical polymerization and more particularly to the formation of the hydrophobic segment of formula (I), the oligo- or polysaccharide is completely soluble in the medium defined by the solvent.

Insofar as it is desired to favor the formation of the copolymer directly in the form of particles or of micelles, the solvent is also chosen in order to be weakly solubilizing or non-solubilizing with respect to the copolymer.

Preferably, the poly- or oligosaccharide molecule is chosen from dextran, heparin, poly(N-acetylneuraminic acid), amylose, chitosan, pectin and hyaluronic acid, and their derivatives.

The solvent is, of course, also chosen in order to remain inert with respect to the polymerization.

Advantageously, such a solvent is preferably chosen from aqueous, aqueous/alcoholic or aqueous/acetone solvents.

The chosen solvent is acidified with an organic or inorganic acid and preferably a nitric acid in order to obtain a pH suitable for the progression of the radical polymerization.

As regards the respective amounts of oligo- or polysaccharide and monomer of general formula (II), they can vary widely. It is a specific advantage of the claimed process to make possible control of the structure of the copolymer which it is desired to prepare. The amounts of reactants introduced are also dependent on their respective molecular masses and on their degrees of solubility in the reaction medium.

As regards the redox initiator, they are generally mixtures of organic or inorganic oxidizing agents and reducing agents which generate radicals during the electron transfer stage. This generation of radicals has the advantage of requiring a low activation energy, in contrast to conventional radical initiators, which makes it possible to initiate radical polymerizations at relatively low temperatures (0−50° C.).

The redox initiator used preferably comprises at least one metal salt chosen from Ce⁴⁺, V⁵⁺, Cr⁶⁺ or Mn³⁺ salts. According to a preferred alternative form of the invention, it is a Ce⁴⁺ salt. It is generally introduced in the form of cerium ammonium nitrate.

The concentration of radical initiator is also capable of influencing the progression of the radical polymerization. Thus it is that the composition of the copolymer and the length of the respective blocks of the polysaccharide and of the polymer of general formula (I) can be varied according to the concentration of initiator. Its adjustment comes within the competence of a person skilled in the art.

As regards the reaction temperature, it is adjusted to a value compatible with the initiation of the polymerization.

This temperature is generally between 0 and 50° C.

As regards the order of introduction of the various reactants, it is preferable to dissolve the oligo- or polysaccharide in the chosen solvent and then to add the redox radical initiator. The monomer of formula (II) is subsequently introduced into the mixture.

On conclusion of the process, the copolymer can be obtained in a soluble form or in the form of micelles, powders or particles. It is preferably obtained directly in the form of particles. The particles can be charged with active materials either after their preparation or during their preparation.

When the copolymer is obtained in the form of a powder, it is, of course, possible to formulate this powder in the form of particles by using appropriate conversion techniques. Mention may more particularly be made, by way of illustration of these techniques, of emulsification/solvent evaporation, emulsification/solvent diffusion or nanoprecipitation techniques.

The particles correspond to the characteristics set out above.

According to an alternative form of the invention, the polymerization of the compound of general formula (II) is carried out in the presence of the active material to be charged.

At the end of the polymerization, it is possible, if necessary, to neutralize the pH of the reaction medium. Preferably, the latter is adjusted to a value which remains less than or equal to 7.5. The copolymer is recovered by conventional techniques.

According to a preferred alternative form of the invention, the metal salt resulting from the reaction of the radical initiator is complexed, prior to the isolation of the copolymer. This complexing, which comes within the competence of a person skilled in the art, makes it possible to remove these metal salts.

The examples and figures which appear below are presented by way of illustration and without limitation of the present invention.

### FIGURES

FIG. 1: Kinetics of radical polymerizations according to the invention of isobutyl cyanoacrylate in the presence of dextran, of chitosan or of pectin.

FIG. 2: Reference kinetics of anionic polymerizations of isobutyl cyanoacrylate in the presence of dextran or of chitosan.

FIG. 3: Electron paramagnetic resonance (EPR) spectrum of dextran-poly(isobutyl cyanoacrylate) labeled with 4-amino-TEMPO copolymers in accordance with the invention.

Equipment and Method

The size of the polymer particles (mean hydrodynamic diameter) is determined using a nanosizer (Coulter N4 Plus®) by quasi-elastic scattering of laser radiation.

The surface charge of the particles is determined using a Zetasizer 4 Malvern®. To do this, the dextranpoly(isobutyl cyanoacrylate) and heparin-poly(isobutyl cyanoacrylate) suspensions are diluted respectively to 1/200th and 1/30th in 1 mmol/l potassium chloride.

### EXAMPLE 1

0.1375 g of dextran 70000 g/mol are dissolved in 8 ml of HNO₃ (0.2 mol/l) in a glass tube with a diameter of 2 cm with magnetic stirring at 40° C. and with slight bubbling with argon. After 10 minutes, 2 ml of acid solution of cerium ions (8×10⁻² mol/l of cerium(IV) ammonium nitrate in 0.2 mol/l HNO₃) and then 0.5 ml of isobutyl cyanoacrylate are added. After 10 minutes, the bubbling of argon is halted and the glass tube is stoppered. After at least 40 min, stirring is halted and the glass tube is cooled under mains water. The pH is adjusted with NaOH (1N) in order, after the addition of 1.25 ml of trisodium citrate dihydrate (1.02 mol/1), for it to arrive directly at a value of 7±0.5. Finally, the suspension is stored in a refrigerator.

At this stage, a suspension of stable colloidal polymer particles is obtained. The particles of copolymers can then be purified.

The particles of copolymers are purified as follows:

Dialysis bags (Spectra/Por® CE MWCO: 100000) are regenerated for 30 minutes with osmosed water, and the colloidal suspensions are vortex mixed and then introduced into the bags.

Two successive dialyses lasting 1 h 30 are carried out against 5 liters of osmosed water, which are followed by another dialysis overnight against 5 liters of osmosed water.

The suspensions of particles of copolymers, thus purified and present in the dialysis bags, are recovered and then stored at (+4° C.) or optionally dried by freeze drying.

The freeze drying of the particles of copolymers is carried out as follows:

The freeze dryings are carried out without the addition of cryoprotective agent.

The suspensions of colloidal particles are divided into aliquots in sample tubes and then frozen (−18° C.). The lyophilization (Bioblock Scientific Christ alpha 1-4) is carried out for 48 hours. The lyophilizates (white powders) are stored in a refrigerator.

Reconstitution of the Particles:

To reconstitute the dispersion of particles from the lyophilizate, a predetermined mass of lyophilizate is dispersed in a known volume of MilliQ® water in order for the mass of lyophilizate/volume of water ratio to be 1%. The suspension is homogenized using a vortex mixer at the maximum speed and then by ultrasound for a few minutes using an ultrasonic bath (Branson 5200®).

### EXAMPLE 2

The same protocol as that described in example 1 is repeated using, instead of dextran 70000 g/mol, one of the following polysaccharides:

0.1375 g of heparin;

0.0230 g of chitosan;

0.0230 g of pectin;

a soluble amount of hyaluronic acid in 8 ml of HNO₃ (0.2 mol/l);

0.1375 g of dextran sulfate with a molecular mass (6-8000, 10000, 40000, 50000 or 500000 g/mol);

0.1375 g of γ-cyclodextrin; or

0.1375 g of dextran 15-20000 g/mol.

### EXAMPLE 3

The protocol of example 1 is repeated, one of the following monomers being substituted for isobutyl cyanoacrylate:

isohexyl cyanoacrylate,

n-butyl cyanoacrylate,

n-propyl cyanoacrylate,

ethyl cyanoacrylate, or

2-methoxyethyl cyanoacrylate.

### EXAMPLE 4

Physical Characterization of the Particles:

The copolymers' obtained in examples 1, 2 and 3 are characterized in terms of size, stability and charge.

Size of the Suspensions:

The suspensions were diluted beforehand with MilliQ® water.

The sizes of the particles of the various particulate suspensions prepared are summarized in table 1 below.

The stability of the suspensions obtained was evaluated as a function of the size of the particles over time. The results obtained appear in table 2 below.

TABLE 1

| Polysaccharides | | Alkyl cyanoacrylate monomers: derivatives | | | | | |
|---|---|---|---|---|---|---|---|
| Nature | Mass* (g) | isohexyl | isobutyl | n-butyl | n-propyl | ethyl | 2-methoxyethyl |
| Dextran 70000 | 0.1375 | 236 ± 3 nm | 290 ± 2 nm | 227 ± 3 nm | 275 ± 2 nm | 443 ± 7 nm | 375 ± 10 nm |
| Dextran 15-20000 | 0.1375 | — | 170 ± 2 nm | — | — | — | — |
| Heparin | 0.1375 | — | 87 ± 2 nm | — | — | — | — |
| Chitosan | 0.0230 | — | ≧1 μm | — | — | — | — |
| Pectin | 0.0230 | — | ≧1 μm | — | — | — | — |
| Hyaluronic acid | S** | — | ≧1 μm | — | — | — | — |
| Dextran sulfate | 0.1375 | — | 408 ± 4 nm | — | — | — | — |
| 500000 | | | | | | | |
| Dextran sulfate | 0.1375 | — | 333 ± 4 nm | — | — | — | — |
| 50000 | | | | | | | |
| Dextran sulfate | 0.1375 | — | 274 ± 2 nm | — | — | — | — |
| 40000 | | | | | | | |
| Dextran sulfate | 0.1375 | — | 192 ± 2 nm | — | — | — | — |
| 10000 | | | | | | | |
| Dextran sulfate | 0.1375 | — | 805 ± 10 nm | — | — | — | — |
| 6-8000 | | | | | | | |
| γ-Cyclodextrin | 0.1375 | — | aggregates | — | — | — | — |

*mass of polysaccharide dissolved in 8 ml of 0.2 mol/l nitric acid.
**mass soluble in 8 ml of 0.2 mol/l nitric acid.
The standard deviations represent the repeatability of the measurements.

TABLE 2

| | Products | |
|---|---|---|
| | Dextran- | Heparin- |
| | poly(isobutyl | poly(isobutyl |
| Time | cyanoacrylate) | cyanoacrylate) |
| T₀ | 290 ± 2 nm | 87 ± 2 nm |
| T₀ + dialysis | 297 ± 3 nm | 93 ± 2 nm |
| T₀ + dialysis, storage for 1 month | 282 ± 4 nm | 113 ± 10 nm |
| T₀ + dialysis, storage for 6 months | 290 ± 2 nm | 102 ± 3 nm |
| T₀ + dialysis, storage for 30 months | 286 ± 5 nm | 85 ± 2 nm |
| T₀ + dialysis + freeze drying | 312 ± 7 nm | — |
| Storage for 6 months | | |
| Redispersion + ultrasound treatment | | |
| T₀ + dialysis + freeze drying | 296 ± 3 nm | — |
| Storage for 14 months | | |
| Redispersion + ultrasound treatment | | |

T₀: day of the preparation.
The standard deviations represent the repeatability of the measurements.

Zeta Potential:

The results obtained are reported in table 3 below.

TABLE 3

| | Zeta | Standard |
|---|---|---|
| | potential | deviations |
| Suspensions | (mV) | (mV) |
| Dextran-poly(isobutyl | −10.6 | 0.9 |
| cyanoacrylate) | | |
| Heparin-poly(isobutyl | −36.1 | 1.4 |
| cyanoacrylate) | | |

It should be noted that the Zeta potential obtained with the heparin-poly(isobutyl cyanoacrylate) particles is further from neutrality than that obtained with the dextran-poly(isobutyl cyanoacrylate) particles.

These results thus reproduce the difference in natural charge between the heparin and the dextran. Obviously, the polysaccharide present in each of the two types of particles is located at the surface of the latter.

### EXAMPLE 5

Characterization of the Content of Copolymer in the Suspensions and Composition of the Copolymers:

The content of polymer in the suspensions is determined by evaluation of the weight of the dry residue obtained after freeze drying a known amount of suspension purified by dialysis. To do this, an aliquot of purified suspension prepared according to example 1 or 2 is accurately weighed in a sample tube and then frozen to (−18° C.) before freeze drying for 48 h in a Christ Alpha 1-4 freeze dryer (Bioblock Scientific). The mass of lyophilizate is weighed and then related back to the mass of initial suspension.

The suspension of dextran-poly(isobutyl cyanoacrylate) copolymer obtained according to example 1 comprises 3.1±0.4% of copolymer (mass/mass).

The suspension of heparin-poly(isobutyl cyanoacrylate) copolymer obtained according to example 2 comprises 2.4±0.7% of copolymer (mass/mass).

The composition of the copolymers is evaluated by elemental analysis of the powders obtained by freeze drying the purified suspensions as is indicated above. The dextran-poly(isobutyl cyanoacrylate) copolymer obtained according to example 1 comprises 20% (mass/mass) of dextran.

### EXAMPLE 6

Kinetics of Polymerization

Equipment and Apparatus:

Spectrometer of “PC2000 Plug-in” type (Ocean Optics Europe) inserted in a computer of PC type, an HL-2000-LL light source (Ocean Optics Europe), optical fibers (200 and 100 μm) (Top sensor systems FC-UV, Ocean Optics Europe) and OOI Base V 1.5 software (Ocean Optics Europe).

Round-bottomed glass tube with a diameter of 2 cm for carrying out the polymerization.

Teflon® collar with holes at 0°, 90° and 180°. The size of the holes is adjusted in order to act as support for the optical fibers and the size of the Teflon® collar is adjusted to the glass tube in which the polymerization will be carried out. The optical fibers are fitted to the collar in the 0° and 1800 positions for absorbance measurements.

In this test, the kinetics of radical polymerization of isobutyl cyanoacrylate in the presence of dextran, chitosan or pectin were monitored.

The polymerization is carried out according to the protocol described in examples 1 to 3 in the glass tube with a diameter of 2 cm placed in a water bath at 40° C. and on which the Teflon® collar supporting the optical fibers connected to the spectrometer and to the light source is fitted. The bubbling with argon is positioned so as not to interfere with the acquisition of the measurements. The background noise of the spectrometer is recorded before the introduction of the acid solution of cerium(IV) ions (8×10⁻² mol/l of cerium ammonium nitrate in 0.2 mol/l HNO₃). The reference is recorded after the addition of the acid solution of cerium(IV) ions (8×10⁻² mol/l of cerium ammonium nitrate in 0.2 mol/l HNO₃). The recording of the polymerization kinetics is begun from the addition of the 0.5 ml of monomer. It is carried out by the quasi-instantaneous acquisition of an absorbence spectrum over a broad wavelength range (400-800 nm) every 30 seconds for 50 min. The absorbances measured at the wavelength of 650 nm are used to plot curves of absorbance as a function of time, thus reflecting the kinetics of polymerization.

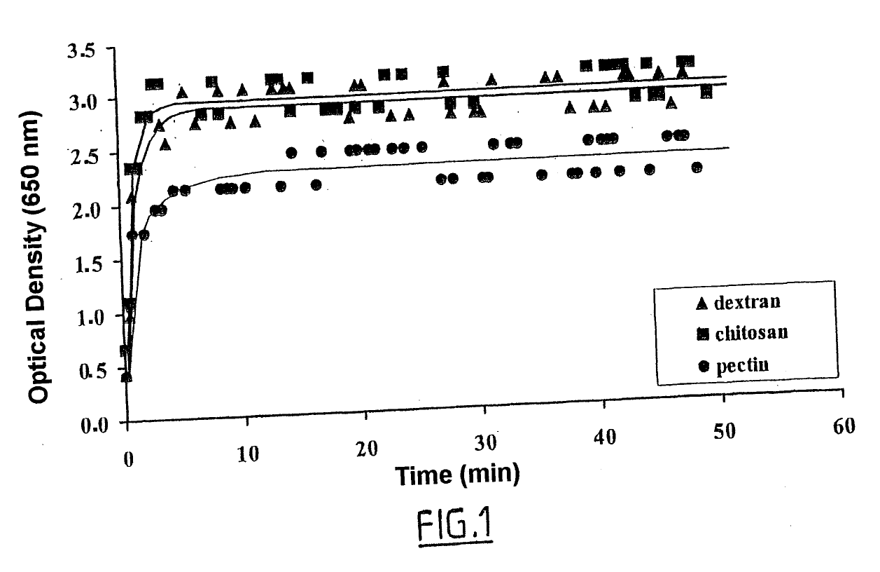

The results are presented in FIG. 1.

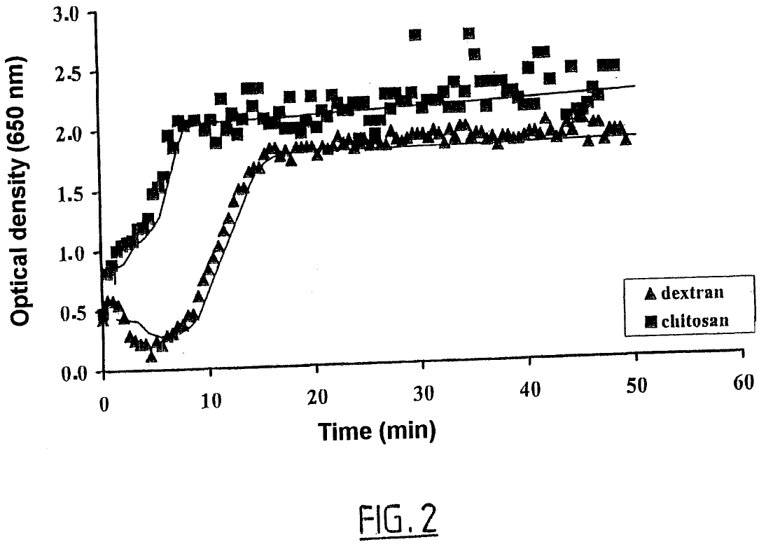

The kinetics of an anionic polymerization carried out with the same monomer in the presence of dextran or of chitosan but in the absence of the redox initiator responsible for the radical polymerization are reported in FIG. 2, by way of comparison.

It should be noted that the initiation of the anionic polymerization is delayed in comparison with the radical polymerization.

### EXAMPLE 7

This example illustrates the synthesis of dextranpoly(isobutyl cyanoacrylate) particles on a greater scale.

0.6875 g of dextran 70000 g/mol are dissolved in 40 ml of 0.2 mol/l nitric acid in a 50 ml flat-bottomed ground-neck flask with magnetic stirring at 40° C. and with gentle bubbling of argon for 10 min. An acid solution of cerium ions (10 ml) (8×10⁻² mol/l of cerium(IV) ammonium nitrate in 0.2 mol/l nitric acid) and then 2.5 ml of isobutyl cyanoacrylate are then added and kept vigorously stirred while maintaining bubbling of argon for a further 10 min. The bubbling of argon is halted and the flask is stoppered. The reaction is continued with stirring at 40° C. for 50 min. The reaction is halted and the flask is cooled under mains water. The pH is adjusted with NaOH (1N) in order, after the addition of 6.25 ml of trisodium citrate dihydrate (1.02 mol/l), for it to arrive directly at a value of 7±0.5. The mean hydrodynamic diameter of the particles of copolymers obtained is 291±1 nm.

### EXAMPLE 8

This example illustrates the synthesis of dextranpoly(isobutyl cyanoacrylate) particles according to simplified experimental conditions.

0.1375 g of dextran 70000 g/mol are dissolved in 8 ml of HNO₃ (0.2 mol/l) in a 20 ml screw-capped bottle with magnetic stirring at 20° C. After 10 minutes, 2 ml of acid solution of cerium ions (8×10⁻² mol/l of cerium(IV) ammonium nitrate in 0.2 mol/l HNO₃) and then 0.5 ml of isobutyl cyanoacrylate are added. After 60 minutes, stirring is halted. The pH is adjusted with NaOH (1N) in order, after the addition of 1.25 ml of trisodium citrate dihydrate (1.02 mol/l), for it to arrive directly at a value of 7±0.5. The mean hydrodynamic diameter of the particles of copolymers obtained is 393±5 nm.

### EXAMPLE 9

Evaluation of the Residual Anticoagulant Biological Activity of the Heparin of the Heparin-Poly(Isobutyl Cyanoacrylate) Copolymer:

The anticoagulant activity of the heparin of the heparin-poly(isobutyl cyanoacrylate) copolymer is evaluated by measuring the activated cephalin time (ACT) or anti-IIa activity and by measuring the anti-Xa activity produced by the particles composed of said copolymer synthesized according to example 2.

Measurement of the Anti-IIa Activity:

Frozen normal plasma is defrosted in a water bath at 37° C. and then placed in an ice tray. The APTT reagent (Organon Teknica Corporation, Fresnes, France) is regenerated with 3 ml of sterile water. A 1/40 mol/1 CaCl₂ solution is prepared in Owren-Koller buffer (OKB) (Diagnostic Stago).

Preparation of the Samples:

A range for calibrating the method is prepared with the same heparin as that used for the synthesis of the copolymer. A 1700 IU/ml mother solution is prepared in the OKB buffer and is then diluted in this same buffer to give 0.17, 0.85, 1.7, 4.25 and 8.5 IU/ml solutions. 100 μl of each of the dilutions are themselves diluted in 900 μl of normal plasma.

The suspensions of particles of copolymers are also diluted in the OKB to 1/100th and to 1/200th. 100 μl of each of the dilutions of the suspension are themselves diluted in 900 μl of normal plasma.

A clotting control is composed of 100 μl of OKB and 900 μl of normal plasma.

Measurement of the Clotting Times:

A bead is placed in each of the cells of the ST4 coagulometer (Diagnostica Stago) and then 100 μl of one of the samples prepared in the preceding stage and 100 μl of the APTT solution are introduced into the various cells. After incubating for 300 seconds at 37° C., 100 μl of the calcium chloride solution are added. The coagulometer measures the clotting times of the various samples in seconds. The results obtained for the control heparin solutions make it possible to draw up a calibration curve giving the activity of the heparin solution, expressed in IU/ml, as a function of the clotting time, expressed in seconds. The activity of the heparin associated with the copolymer particles is evaluated on the calibration curve from the clotting times measured for the suspensions.

Thus, the suspension comprising particles, prepared according to example 2, of heparin-poly(isobutyl cyanoacrylate) copolymers exhibit an anti-IIa activity of 329±28 IU/ml.

Measurement of the Anti-Xa Activity:

The suspension of copolymer particles is diluted to 1/50th, to 1/100th and to 1/200th in the OKB buffer and then to 1/10th in defrosted normal plasma as indicated above. The clotting times are evaluated automatically on an ST1 coagulometer (Diagnostica Stago) automatically.

The anti-Xa activity of the suspension of particles of the heparin-poly(isobutyl cyanoacrylate) copolymers is 408±50 IU/ml.

### EXAMPLE 10

Grafting of a Label to Particles of Copolymers:

A suspension, not purified by dialysis, of particles of copolymers is prepared according to example 2 with dextran 15-20000 g/mol. The suspension is filtered through a 1.2 μm filter (Millipore® SLA P0 2550) and then purified by 2 dialyses of 2 hours against 1 l of osmosed water, followed by one dialysis of 2 hours against 1 l of phosphate buffer (Sigma ref. P 3813) (dialysis membrane: Spectra/Por® CE MWCO: 100000 regenerated for 30 min in osmosed water).

For the grafting, 0.0270 g of 1,1′-carbonyldiimidazole (Sigma ref. C-7625) and 0.0113 g of 4-amino-TEMPO (Aldrich) are introduced into a 20 ml screw-capped flask equipped with a cap. These products are dissolved in 0.5 ml of phosphate buffer with stirring. 3 ml of the purified suspension of particles of copolymers are added to this mixture. The combined mixture is kept stirred magnetically at ambient temperature for 48 hours. After the reaction, the excess reactants and the reaction byproducts are removed by dialysis. The suspension is placed in a dialysis bag (Spectra/Por® CE MWCO: 100000), regenerated beforehand for 30 min with osmosed water, and then dialyzed three times against 1 l of phosphate buffer for 2 hours. The suspensions of grafted particles are recovered and can be stored at (+4° C.).

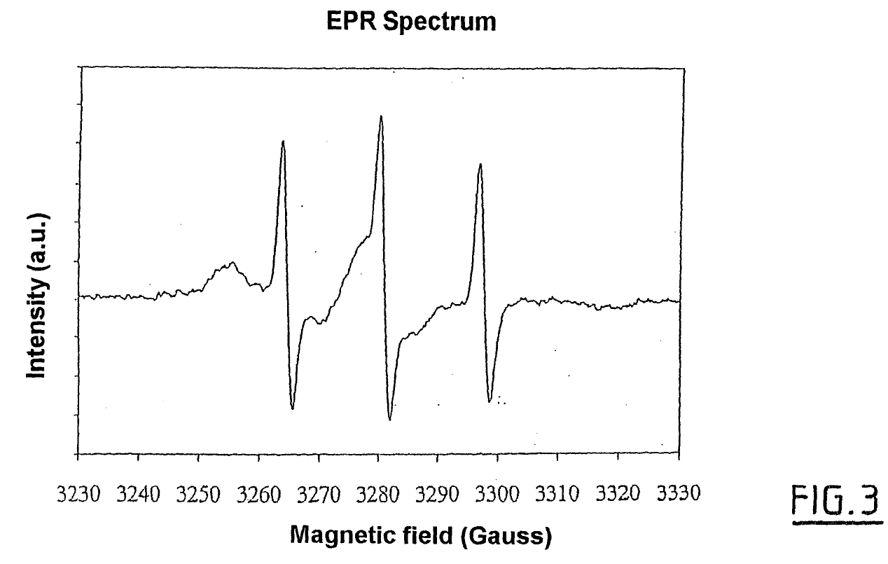

The grafting of the label can be demonstrated by electron paramagnetic resonance (EPR) spectroscopy. To do this, the suspension obtained is placed in a measuring cell of a Varian E-4 EPR spectrometer. The spectrum obtained, presented in FIG. 3, indicates that the 4-amino-TEMPO has indeed been grafted to the dextran chains of the copolymer forming the particles and that it is 81% found under slow motion conditions and 19% found under fast motion conditions according to the Kivelson simulation (Kivelson D. J., Journal Chem. Phys., 1960, 33, 1107).

1. A copolymer comprising a block structure composed of a hydrophilic segment of saccharide nature and at least one biodegradable hydrophobic segment of general formula (I) 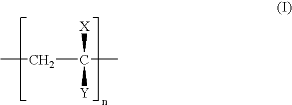
in which:
X represents a CN or CONHR radical,
Y represents a COOR′ or COHNR″ radical,
with R, R′ and R″ representing, independently of one another, a hydrogen atom, a linear or branched C₁ to C₂₀ alkyl group, a linear or branched C₁ to C₂₀ alkoxy group, an amino acid radical, a mono- or polyhydroxylated acid radical or a C₅ to C₁₂ aryl or heteroaryl radical,
with said segment of saccharide nature being bonded either by one of its ends to a single segment of general formula (I) or by each of its two ends to a segment of general formula (I), the two hydrophobic segments being identical or different.

2. The copolymer as claimed in claim 1, characterized in that X represents in general formula (I) a CN radical.

3. The copolymer as claimed in claim 1 or 2, characterized in that Y represents in general formula (I) COOR′ with R′ as defined in claim 1.

4. The copolymer as claimed in one of the preceding claims, characterized in that the segment of saccharide nature derives from a natural or synthetic oligo- or polysaccharide which may or may not be modified.

5. The copolymer as claimed in one of claims 1 to 4, characterized in that the oligo- or polysaccharide has biological properties and/or activities.

6. The copolymer as claimed in one of claims 1 to 5, characterized in that the oligo- or polysaccharide is chosen from polydextroses, such as dextran, chitosan, pullulan, starch, amylose, cyclodextrins, hyaluronic acid, heparin, amylopectin, cellulose, pectin, alginate, curdlan, fucan, succinoglycan, chitin, xylan, xanthan, arabinan, carrageenan, poly(glucuronic acid), poly(N-acetylneuraminic acid), poly(mannuronic acid) and their derivatives.

7. The copolymer as claimed in claim 6, characterized in that it is dextran, heparin, poly(N-acetylneuraminic acid), amylose, chitosan, pectin, hyaluronic acid, or one of their derivatives.

8. The copolymer as claimed in one of claims 1 to 7, characterized in that it is obtained by radical polymerization of at least one molecule of a compound of general formula (II): 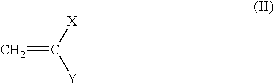
in which X and Y are as defined in claim 1, 2 or 3, in the presence of a poly- or oligosaccharide.

9. The copolymer as claimed in claim 8, characterized in that that the radical polymerization is carried out at pH and atmosphere conditions unfavorable to the presence and/or to the generation of anions in the reaction medium and in the presence of a sufficient amount of a suitable redox radical initiator.

10. The copolymer as claimed in one of the preceding claims, characterized in that it is provided under the appearance of particles.

11. A particle, characterized in that it is composed of a copolymer as claimed in one of claims 1 to 9.

12. The particle as claimed in claim 11, characterized in that it is composed of a copolymer deriving from the polymerization of:
isohexyl cyanoacrylate, isobutyl cyanoacrylate, n-butyl cyanoacrylate, n-propyl cyanoacrylate, ethyl cyanoacrylate or 2-methoxyethyl cyanoacrylate in the presence of dextran, or
isobutyl cyanoacrylate in the presence of heparin, chitosan, pectin, hyaluronic acid, dextran sulfate or γ-cyclodextrin.

13. The particle as claimed in claim 11 or 12, characterized in that it exhibits a size of between 1 nm and 1 mm.

14. The particle as claimed in one of claims 11 to 13, characterized in that it incorporates a biological or pharmaceutical material.

15. Use of particles as claimed in one of claims 11 to 14, as vehicle for pharmaceutical, food-processing, cosmetic or veterinary active principles.

16. A process of use in the preparation of block copolymers composed of a hydrophilic segment of saccharide nature, at least one of the ends of which is bonded to a hydrophobic segment, characterized in that it comprises the polymerization by the radical route of at least one molecule of a compound of general formula (II): 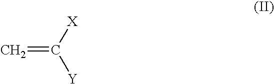
in which:
X represents a CN or CONHR radical,
Y represents a COOR′ or COHNR″ radical,
with R, R′ and R″ representing, independently of one another, a hydrogen atom, a linear or branched C₁ to C₂₀ alkyl group, a linear or branched C₁ to C₂₀ alkoxy group, an amino acid radical, a mono- or polyhydroxylated acid radical or a C₅ to C₁₂ aryl or heteroaryl radical,
said radical polymerization being carried out in the presence of at least one molecule of a poly- or oligosaccharide under pH and atmospheric conditions unfavorable to the presence and/or to the generation of anions in the reaction medium and in the presence of a sufficient amount of a suitable radical redox initiator.

17. The process as claimed in claim 16, characterized in that, in the derivative of formula (II), X represents a CN radical and/or Y represents a COOR′ radical with R′ as defined in claim 16.

18. The process as claimed in claim 16 or 17, characterized in that the radical polymerization is carried out at a pH of less than 2 and preferably of less than 1.5.

19. The process as claimed in any one of claims 16 to 18, characterized in that the radical polymerization is carried out in a solvent in which the oligo- or polysaccharide is in the soluble form and the expected copolymer is weakly soluble or insoluble.

20. The process as claimed in one of claims 16 to 19, characterized in that the poly- or oligosaccharide molecule is chosen from dextran, heparin, poly(N-acetylneuraminic acid), amylose, chitosan, pectin and hyaluronic acid, and their derivatives.

21. The process as claimed in one of claims 16 to 20, characterized in that the redox radical initiator comprises at least one metal salt chosen from Ce⁴⁺, V⁵⁺, Cr⁶⁺ or Mn³⁺ salts and preferably a Ce⁴⁺ metal salt.

22. The process as claimed in one of claims 16 to 21, characterized in that the poly- or oligosaccharide is dissolved in the solvent, the redox radical initiator is added and then the monomer of general formula (II) is added.

23. The process as claimed in one of claims 16 to 22, characterized in that the polymerization is carried out in the presence of an active material to be charged to said particles.

24. The process as claimed in one of claims 16 to 23, characterized in that the copolymer is isolated from the reaction medium after neutralization of the pH of the reaction medium.

25. The process as claimed in one of claims 16 to 24, characterized in that the metal salt resulting from the reaction of the radical initiator is complexed, prior to the isolation of the copolymer.

26. The process as claimed in one of claims 16 to 25, characterized in that the copolymer is obtained in the form of particles, of aggregates and/or micelles.
